(19) United States
(12) Patent Application Publication
(10) Pub. No.: US 2024/0310376 A1
(43) Pub. Date: Sep. 19, 2024

(54) Detecting SARS-CoV-2 and Other Infective Agents

(71) Applicant: FLOWMETRIC LIFE SCIENCES, INC, Doylestown, PA (US)

(72) Inventors: Patrick Romano, Doylestown, PA (US); Julie Ann Bick, Easton, PA (US); Renold Julius Capocasale, Mount Laurel, NJ (US)

(21) Appl. No.: 18/274,551

(22) PCT Filed: Jan. 28, 2022
(86) PCT No.: PCT/US22/14301

§ 371 Date: Jul. 27, 2023

Related U.S. Application Data
(60) Provisional application No. 63/143,211, filed on Jan. 29, 2021.

(51) Int. Cl. G01N 33/569; G01N 33/543

(52) U.S. Cl. CPC G01N 33/56983; G01N 33/54313; G01N 2333/165; G01N 2469/10

(57) ABSTRACT

Described herein are compositions for detecting an antigen, including but not limited to, a SARS-CoV-2 antigen, in a sample using a flow cytometry platform. The use of the flow cytometry platform described herein allows for the detection of one or more antibodies in a sample from a patient.

### CROSS-REFERENCE TO RELATED APPLICATIONS

This application is the United States national phase of International Application No. PCT/US22/14301 filed Jan. 28, 2022, and claims priority to U.S. Provisional Application No. 63/143,211 filed Jan. 29, 2021, the disclosures of which are hereby incorporated by reference in their entireties.

### SEQUENCE LISTING

The Sequence Listing associated with this application was filed in electronic format via EFS-Web and is hereby incorporated by reference into the specification in its entirety. The name of the text file containing the Sequence Listing is 9255-2003396_ST25.txt. The size of the text file is 47,881 bytes, and the text file was created on Jan. 20, 2022.

### FIELD OF THE INVENTION

The present disclosure relates to a composition for detecting an antigen, for example, a severe acute respiratory syndrome coronavirus 2 (SARS-CoV-2) antigen, the composition having the antigen conjugated to a capture bead. The present disclosure further relates to a method for detecting an infective agent by flow cytometry including establishing a fluid stream, adding a sample from a patient having one or more antibodies, selecting one or more capture beads conjugated to a protein, incubating the sample and the one or more capture beads with a capture antibody having a detection molecule, and detecting the sample and the one or more capture beads by quantifying the capture antibody, wherein the protein is an antigen, and wherein the antigen, for example, is a SARS-CoV-2 antigen.

### SUMMARY OF THE INVENTION

Coronavirus disease (COVID-19), caused by severe acute respiratory syndrome coronavirus 2 (SARS-CoV-2), has spread rapidly across the world and has affected millions of people worldwide. SARS-CoV-2 has a wide range of hosts including, but not limited to, human, mammals, and birds. Those infected with SARS-CoV-2 may not exhibit symptoms and may remain asymptomatic, while others infected may have severe symptoms in their respiratory and digestive organs.

SARS-CoV-2 is an RNA virus that about 27-32 kb of positive-sense single-stranded RNA. SARS-CoV-2 has at least six open reading frames (ORFs) and may other accessory genes. At the 5′ terminal, about two-thirds of the genome has two ORFs, ORF1 and ORF2. These ORF encode two polyproteins, pp1a and pp1ab, and further cleave into 11 and 16 proteins, respectively. At the 3′ terminal, the various structural proteins are located including, but not limited to, nucleocapsid protein, membrane protein, envelope protein, and spike protein. The spike protein is believed to comprise trimeric spike protein, spike glycoprotein, S1 protein, and S2 protein. Further, the S1 protein comprises an N-terminal domain (NTD) and a receptor binding domain (RBD). SARS-CoV-2 also has accessory proteins which may contribute to SARS-CoV-2 replication. It is believed that spike protein plays an important role in host specificity and receptor binding. SARS-CoV-2 may also contain hemagglutinin-esterase dimer protein in its virion.

Patients infected with SARS-CoV-2 have clinical manifestations which may include, but are not limited to, fever, cough, and shortness of breath. Some patients may have serious complications such as acute respiratory distress syndrome (ARDS) and/or cytokine storm which may lead to death.

Early and accurate testing of SARS-CoV-2 is very important to provide timely medical help to an infected individual as well as help prevent SARS-CoV-2 community spread. False negative test results may lead to the spread of SARS-CoV-2. Similarly, false positive test results may lead to unnecessary treatment and mental trauma to the patients. Therefore, there is a need to have accurate, rapid, readily available and reliable testing for SARS-CoV-2. To date, various immunological and nucleic acid amplification tests have been developed.

Immunological tests measure the antibodies generated by host body's immune response against the virus infection or measures the proteins of SARS-CoV-2 present in the patient's sample. As SARS-CoV-2 enters in the patient, the virus elicits an immune response to produce antibodies (e.g., IgM and/or IgG antibodies) against SARS-CoV-2 protein. Detection of these antibodies in a patient having SARS-CoV-2 is very useful whether or not the person is asymptomatic. Further, detection of these antibodies in a patient following SARS-CoV-2 vaccination is useful for determining vaccine effectiveness and/or evaluating immunological protection following vaccination.

Commercial antibody tests currently on the market typically only test for one of the viral proteins associated with SARS-CoV-2 (i.e., one of either, spike protein, nucleocapsid protein, membrane protein, or envelope protein). Recent reports suggest that while serological tests that measure antibodies to nucleocapsid protein, which is considered to be the most abundant SARS-CoV-2 protein, might be the most sensitive, measuring antibodies to the receptor binding domain (RBD) of the S1 region of the spike protein might be more specific to an anti-viral response.

Accordingly, there is a need to provide a serological test that measures multiple SARS-CoV-2 antigens within the same patient sample for improved specificity and sensitivity. Further, there is a need to provide a serological test that measures SARS-CoV-2 vaccine effectiveness by measuring one or more SARS-CoV-2 antigen(s) within the patient. As SARS-CoV-2 vaccines are engineered to represent and/or mimic various viral antigenic regions of the virus, the serologically evaluation of antibodies, as described herein, targeting these vaccine specific antigenic regions in comparison with viral antigen(s) that are not represented in the vaccine can be used to assess a patient's immune response to vaccination and/or distinguish the patient's immune response from natural infection.

An object of certain embodiments of the present disclosure is to provide a composition for detecting one or more antigens. In some embodiments, the composition may include the antigen conjugated to a capture bead. In further embodiments, the antigen is a severe acute respiratory syndrome coronavirus 2 (SARS-CoV-2) antigen which may include a spike protein, a nucleocapsid protein, an envelope protein, a membrane protein, a hemagglutinin-esterase dimer protein, or an angiotensin-converting enzyme 2 (ACE2) protein. In some non-limiting embodiments, the spike protein may include a S1 protein, a S2 protein, a trimeric spike protein, or a spike glycoprotein. In further embodiments, the S1 protein may include an N-terminal domain (NTD) or a receptor binding domain (RBD).

In some embodiments, the antigen may comprise an amino acid sequence of SEQ ID NO. 1, an amino acid sequence of SEQ ID NO. 2, an amino acid sequence of SEQ ID NO. 3, an amino acid sequence of SEQ ID NO. 4, an amino acid sequence of SEQ ID NO. 5, an amino acid sequence of SEQ ID NO. 6, an amino acid sequence of SEQ ID NO. 7, an amino acid sequence of SEQ ID NO. 8, an amino acid sequence of SEQ ID NO. 9, or an amino acid sequence of SEQ ID NO. 10.

In further non-limiting embodiments of the current disclosure, the antigen is de-glycosylated. In some embodiments, the antigen is coated or tagged with biotin.

In further embodiments, the capture bead of the composition is a polystyrene bead. In some embodiments, the capture bead is a streptavidin coated polystyrene bead. In some embodiments, the capture bead is coated with streptavidin.

In some embodiments, the capture bead has a size in the range from about 2.0 μm to about 12 μm, from about 3.1 μm to about 12 μm, from about 3.1 μm to about 6.9 μm, or from about 3.1 μm to about 6.8 μm. In further embodiments, the size of the capture bead is 3.1 μm.

In some embodiments, the composition of the present disclosure may include the antigen conjugated to the capture bead wherein the conjugation is a biotin-streptavidin conjugation.

Another object of certain embodiments of the present disclosure is to provide a method for detecting an infective agent by flow cytometry. The method includes establishing a fluid stream. A sample from a patient having one or more antibodies is added to the fluid stream. One or more capture bead(s) conjugated to a protein is selected. The sample and the one or more capture beads are incubated with a capture antibody having a detection molecule. The sample and the one or more capture beads are detected by quantifying the capture antibody. The protein includes an antigen, and the antigen is a severe acute respiratory syndrome coronavirus 2 (SARS-CoV-2) antigen. In some embodiments, the SARS-CoV-2 antigen may be a spike protein, a nucleocapsid protein, an envelope protein, a membrane protein, a hemagglutinin-esterase dimer protein, or an angiotensin-converting enzyme 2 (ACE2) protein.

In some embodiments, the spike protein is a S1 protein, a S2 protein, a trimeric spike protein, or a spike glycoprotein. In further embodiments, the S1 protein may include an N-terminal domain (NTD) or a receptor binding domain (RBD).

In some embodiments, the antigen may comprise an amino acid sequence of SEQ ID NO. 1, an amino acid sequence of SEQ ID NO. 2, an amino acid sequence of SEQ ID NO. 3, an amino acid sequence of SEQ ID NO. 4, an amino acid sequence of SEQ ID NO. 5, an amino acid sequence of SEQ ID NO. 6, an amino acid sequence of SEQ ID NO. 7, an amino acid sequence of SEQ ID NO. 8, an amino acid sequence of SEQ ID NO. 9, or an amino acid sequence of SEQ ID NO. 10.

In further non-limiting embodiments of the current disclosure, the antigen is de-glycosylated. In some embodiments, the antigen is coated or tagged with biotin.

In further embodiments, the capture bead of the composition is a polystyrene bead. In some embodiments, the capture bead is a streptavidin coated polystyrene bead. In some embodiments, the capture bead is coated with streptavidin.

In some embodiments, the capture bead has a size in the range from about 2.0 μm to about 12 μm, from about 3.1 μm to about 12 μm, from about 3.1 μm to about 6.9 μm, or from about 3.1 μm to about 6.8 μm. In further embodiments, the size of the capture bead is 3.1 μm.

In some embodiments, the composition of the present disclosure may include the antigen conjugated to the capture bead wherein the conjugation is a biotin-streptavidin conjugation.

In further embodiments, the sample from the patient having one or more antibodies may comprise cells, microvesicles, blood, serum, plasma, urine, or a combination thereof. In some embodiments, the patient is a human.

In some embodiments, the capture antibody incubated with the sample and the one or more capture beads comprises an anti-human IgM, an anti-human IgG, or a combination thereof.

In further embodiments, the detection molecule of the capture antibody is a fluorophore. In further embodiments, the fluorophore is a R-Phycoerythrin protein. In some embodiments, the capture antibody is conjugated to the detection molecule.

In some embodiments, a method for detecting an infective agent by flow cytometry further includes multiplexing the sample with a plurality of capture beads conjugated to the protein.

Various aspects of the present disclosure may be further characterized by one or more of the following clauses:

Clause 1: A composition for detecting an antigen, the composition comprising the antigen conjugated to a capture bead, wherein the antigen is a severe acute respiratory syndrome coronavirus 2 (SARS-CoV-2) antigen selected from the group consisting of a spike protein, a nucleocapsid protein, an envelope protein, a membrane protein, a hemagglutinin-esterase dimer protein, and an angiotensin-converting enzyme 2 (ACE2) protein.

Clause 2: The composition of clause 1, wherein the SARS-CoV-2 antigen is spike protein.

Clause 3: The composition of clause 1 or 2, wherein the SARS-CoV-2 antigen is nucleocapsid protein.

Clause 4: The composition of any one of clauses 1 to 3, wherein the SARS-CoV-2 antigen is envelope protein.

Clause 5: The composition of any one of clauses 1 to 4, wherein the SARS-CoV-2 antigen is membrane protein.

Clause 6: The composition of any one of clauses 1 to 5, wherein the SARS-CoV-2 antigen is hemagglutinin-esterase dimer protein.

Clause 7: The composition of any one of clauses 1 to 6, wherein the SARS-CoV-2 antigen is angiotensin-converting enzyme 2 (ACE2) protein.

Clause 8: The composition of any one of clauses 1 to 7, wherein the spike protein comprises N-terminal domain (NTD) or a receptor binding domain (RBD).

Clause 9: The composition of any one of clauses 1 to 8, wherein the spike protein is NTD protein.

Clause 10: The composition of any one of clauses 1 to 9, wherein the spike protein is RBD.

Clause 11: The composition of any one of clauses 1 to 10, wherein the antigen comprises an amino acid SEQ ID NO. 1, an amino acid SEQ ID NO. 2, an amino acid SEQ ID NO. 3, an amino acid SEQ ID NO. 4, an amino acid SEQ ID NO. 5, an amino acid SEQ ID NO. 6, an amino acid SEQ ID NO. 7, an amino acid SEQ ID NO. 8, an amino acid SEQ ID NO. 9, or an amino acid SEQ ID NO. 10.

Clause 12: The composition of any one of clauses 1 to 11, wherein the antigen comprises amino acid SEQ ID NO. 1.

Clause 13: The composition of any one of clauses 1 to 12, wherein the antigen comprises amino acid SEQ ID NO. 2.

Clause 14: The composition of any one of clauses 1 to 13, wherein the antigen comprises amino acid SEQ ID NO. 3.

Clause 15: The composition of any one of clauses 1 to 14, wherein the antigen comprises amino acid SEQ ID NO. 4.

Clause 16: The composition of any one of clauses 1 to 15, wherein the antigen comprises amino acid SEQ ID NO. 5.

Clause 17: The composition of any one of clauses 1 to 16, wherein the antigen comprises amino acid SEQ ID NO. 6.

Clause 18: The composition of any one of clauses 1 to 17, wherein the antigen comprises amino acid SEQ ID NO. 7.

Clause 19: The composition of any one of clauses 1 to 18, wherein the antigen comprises amino acid SEQ ID NO. 8.

Clause 20: The composition of any one of clauses 1 to 19, wherein the antigen comprises amino acid SEQ ID NO. 9.

Clause 21: The composition of any one of clauses 1 to 20, wherein the antigen comprises amino acid SEQ ID NO. 10.

Clause 22: The composition of any one of clauses 1 to 21, wherein the antigen is de-glycosylated.

Clause 23: The composition of any one of clauses 1 to 22, wherein the antigen is coated or tagged with biotin.

Clause 24: The composition of any one of clauses 1 to 23, wherein the capture bead is a polystyrene bead.

Clause 25: The composition of any one of clauses 1 to 24, wherein the capture bead is a streptavidin coated polystyrene bead.

Clause 26: The composition of any one of clauses 1 to 25, wherein the capture bead is coated with streptavidin.

Clause 27: The composition of any one of clauses 1 to 26, wherein the capture bead has a size in the range from about 2.0 μm to about 12 μm, from about 3.1 μm to about 12 μm, from about 3.1 μm to about 6.9 μm, or from about 3.1 μm to about 6.8 μm.

Clause 28: The composition of any one of clauses 1 to 27, wherein the capture bead has a size in the range from about 2.0 μm to about 12 μm.

Clause 29: The composition of any one of clauses 1 to 27, wherein the capture bead has a size in the range from about 3.1 μm to about 12 μm.

Clause 30: The composition of any one of clauses 1 to 27, wherein the capture bead has a size in the range from about 3.1 μm to about 6.9 μm.

Clause 31: The composition of any one of clauses 1 to 27, wherein the capture bead has a size in the range from about 3.1 μm to about 6.8 μm.

Clause 32: The composition of any one of clauses 1 to 27, wherein the capture bead has a size of about 2.0 μm.

Clause 33: The composition of any one of clauses 1 to 27, wherein the capture bead has a size of about 3.0 μm.

Clause 34: The composition of any one of clauses 1 to 27, wherein the capture bead has a size of about 4.0 μm.

Clause 35: The composition of any one of clauses 1 to 27, wherein the capture bead has a size of about 5.0 μm.

Clause 36: The composition of any one of clauses 1 to 27, wherein the capture bead has a size of about 6.0 μm.

Clause 37: The composition of any one of clauses 1 to 27, wherein the capture bead has a size of about 7.0 μm.

Clause 38: The composition of any one of clauses 1 to 27, wherein the capture bead has a size of about 8.0 μm.

Clause 39: The composition of any one of clauses 1 to 27, wherein the capture bead has a size of about 9.0 μm.

Clause 40: The composition of any one of clauses 1 to 27, wherein the capture bead has a size of about 10.0 μm.

Clause 41: The composition of any one of clauses 1 to 27, wherein the capture bead has a size of about 11.0 μm.

Clause 42: The composition of any one of clauses 1 to 27, wherein the capture bead has a size of about 12.0 μm.

Clause 43: The composition of any one of clauses 1 to 27, wherein the capture bead is 3.1 μm.

Clause 44: The composition of any one of clauses 1 to 43, wherein the conjugation is a biotin-streptavidin conjugation.

Clause 45: A method for detecting an infective agent by flow cytometry comprising establishing a fluid stream, adding a sample from a patient having one or more antibodies, selecting one or more capture beads conjugated to a protein, incubating the sample and the one or more capture beads with a capture antibody having a detection molecule, and detecting the sample and the one or more capture beads by quantifying the capture antibody, wherein the protein is an antigen, and wherein the antigen is a severe acute respiratory syndrome coronavirus 2 (SARS-CoV-2) antigen selected from the group consisting of a spike protein, a nucleocapsid protein, an envelope protein, a membrane protein, a hemagglutinin-esterase dimer protein, and an angiotensin-converting enzyme 2 (ACE2) protein.

Clause 46: The method of clause 45, wherein the SARS-CoV-2 antigen is spike protein.

Clause 47: The method of clause 45 or 46, wherein the SARS-CoV-2 antigen is nucleocapsid protein.

Clause 48: The method of any one of clauses 45 to 47, wherein the SARS-CoV-2 antigen is envelope protein.

Clause 49: The method of any one of clauses 45 to 48, wherein the SARS-CoV-2 antigen is membrane protein.

Clause 50: The method of any one of clauses 45 to 49, wherein the SARS-CoV-2 antigen is hemagglutinin-esterase dimer protein.

Clause 51: The method of any one of clauses 45 to 50, wherein the SARS-CoV-2 antigen is angiotensin-converting enzyme 2 (ACE2) protein.

Clause 52: The method of any one of clauses 45 to 51, wherein the spike protein comprises N-terminal domain (NTD) or a receptor binding domain (RBD).

Clause 53: The method of any one of clauses 45 to 52, wherein the spike protein is NTD.

Clause 54: The method of any one of clauses 45 to 53, wherein the spike protein is RBD.

Clause 55: The method of any one of clauses 45 to 54, wherein the antigen comprises an amino acid SEQ ID NO. 1, an amino acid SEQ ID NO. 2, an amino acid SEQ ID NO. 3, an amino acid SEQ ID NO. 4, an amino acid SEQ ID NO. 5, an amino acid SEQ ID NO. 6, an amino acid SEQ ID NO. 7, an amino acid SEQ ID NO. 8, an amino acid SEQ ID NO. 9, or an amino acid SEQ ID NO. 10.

Clause 56: The method of any one of clauses 45 to 55, wherein the antigen comprises amino acid SEQ ID NO. 1.

Clause 57: The method of any one of clauses 45 to 56, wherein the antigen comprises amino acid SEQ ID NO. 2.

Clause 58: The method of any one of clauses 45 to 57, wherein the antigen comprises amino acid SEQ ID NO. 3.

Clause 59: The method of any one of clauses 45 to 58, wherein the antigen comprises amino acid SEQ ID NO. 4.

Clause 60: The method of any one of clauses 45 to 59, wherein the antigen comprises amino acid SEQ ID NO. 5.

Clause 61: The method of any one of clauses 45 to 60, wherein the antigen comprises amino acid SEQ ID NO. 6.

Clause 62: The method of any one of clauses 45 to 61, wherein the antigen comprises amino acid SEQ ID NO. 7.

Clause 63: The method of any one of clauses 45 to 62, wherein the antigen comprises amino acid SEQ ID NO. 8.

Clause 64: The method of any one of clauses 45 to 63, wherein the antigen comprises amino acid SEQ ID NO. 9.

Clause 65: The method of any one of clauses 45 to 64, wherein the antigen comprises amino acid SEQ ID NO. 10.

Clause 66: The method of any one of clauses 45 to 65, wherein the antigen is de-glycosylated.

Clause 67: The method of any one of clauses 45 to 66, wherein the antigen is coated or tagged with biotin.

Clause 68: The method of any one of clauses 45 to 67, wherein the capture bead is a polystyrene bead.

Clause 69: The method of any one of clauses 45 to 68, wherein the capture bead is a streptavidin coated polystyrene bead.

Clause 70: The method of any one of clauses 45 to 69, wherein the capture bead is coated with streptavidin.

Clause 71: The method of any one of clauses 45 to 70, wherein the capture bead has a size in the range from about 2.0 μm to about 12 μm, from about 3.1 μm to about 12 μm, from about 3.1 μm to about 6.9 μm, or from about 3.1 μm to about 6.8 μm.

Clause 72: The method of any one of clauses 45 to 71, wherein the capture bead has a size in the range from about 2.0 μm to about 12 μm.

Clause 73: The method of any one of clauses 45 to 71, wherein the capture bead has a size in the range from about 3.1 μm to about 12 μm.

Clause 74: The method of any one of clauses 45 to 71, wherein the capture bead has a size in the range from about 3.1 μm to about 6.9 μm.

Clause 75: The method of any one of clauses 45 to 74, wherein the capture bead has a size in the range from about 3.1 μm to about 6.8 μm.

Clause 76: The method of any one of clauses 45 to 74, wherein the capture bead has a size of about 2.0 μm.

Clause 77: The method of any one of clauses 45 to 74, wherein the capture bead has a size of about 3.0 μm.

Clause 78: The method of any one of clauses 45 to 74, wherein the capture bead has a size of about 4.0 μm.

Clause 79: The method of any one of clauses 45 to 74, wherein the capture bead has a size of about 5.0 μm.

Clause 80: The method of any one of clauses 45 to 74, wherein the capture bead has a size of about 6.0 μm.

Clause 81: The method of any one of clauses 45 to 74, wherein the capture bead has a size of about 7.0 μm.

Clause 82: The method of any one of clauses 45 to 74, wherein the capture bead has a size of about 8.0 μm.

Clause 83: The method of any one of clauses 45 to 74, wherein the capture bead has a size of about 9.0 μm.

Clause 84: The method of any one of clauses 45 to 74, wherein the capture bead has a size of about 10.0 μm.

Clause 85: The method of any one of clauses 45 to 74, wherein the capture bead has a size of about 11.0 μm.

Clause 86: The method of any one of clauses 45 to 74, wherein the capture bead has a size of about 12.0 μm.

Clause 87: The method of any one of clauses 45 to 74, wherein the capture bead is 3.1 μm.

Clause 88: The method of any one of clauses 45 to 87, wherein the conjugation is a biotin-streptavidin conjugation.

Clause 89: The method of any one of clauses 45 to 88, wherein the sample is selected from the group consisting of cells, microvesicles, blood, serum, plasma, urine, and a combination thereof.

Clause 89: The method of any one of clauses 45 to 89, wherein the sample is cells.

Clause 90: The method of any one of clauses 45 to 89, wherein the sample is microvesicles.

Clause 91: The method of any one of clauses 45 to 89, wherein the sample is blood.

Clause 92: The method of any one of clauses 45 to 89, wherein the sample is serum.

Clause 93: The method of any one of clauses 45 to 89, wherein the sample is urine.

Clause 94: The method of any one of clauses 45 to 93, wherein the patient is a human.

Clause 95: The method of any one of clauses 45 to 94, wherein the patient is vaccinated with a SARS-CoV-2 vaccine.

Clause 96: The method of any one of clauses 45 to 95, wherein the capture antibody is selected from the group consisting of an anti-human IgM, an anti-human IgG, and a combination thereof.

Clause 97: The method of any one of clauses 45 to 96, wherein the capture antibody is anti-human IgM.

Clause 98: The method of any one of clauses 45 to 96, wherein the capture antibody is anti-human IgG.

Clause 99: The method of any one of clauses 45 to 98, wherein the detection molecule is a fluorophore.

Clause 100: The method of any one of clauses 45 to 99, wherein the fluorophore is a R-Phycoerythrin protein.

Clause 101: The method of any one of clauses 45 to 100, wherein the capture antibody is conjugated to the detection molecule.

Clause 102: The method of any one of clauses 45 to 101, further comprising multiplexing the sample with a plurality of capture beads conjugated to the protein.

### BRIEF DESCRIPTION OF THE DRAWING(S)

FIG. 1 is an illustration of an embodiment of the method of flow cytometry for detecting an infective agent such as, for example, a SARS-CoV-2 antigen.

FIGS. 2A-2B depict illustrations of a COVID-19 serology test readout pre-SARS-CoV-2 infection and a timeline of the antibody response post-SARS-CoV-2 infection.

FIGS. 3A-3B depict illustrations of a T-cell activation assay test readout demonstrating the secretion of IFN-γ and TNF-α by T-cells in response to SARS-CoV-2 antigen exposure in vitro.

### DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” diluent, “an” intragranular excipient, “a” disintegrant, and the like refer to one or more of any of these items.

“About” as used herein means ±10% of the referenced value. In certain embodiments, “about” means ±9%, or ±8%, or ±7%, or ±6%, or ±5%, or ±4%, or ±3%, or ±2% or ±1% of the referenced value.

One object of the invention disclosed herein is to provide a flow cytometry platform for the serological testing of one or more viral or bacterial antigens. Antigens may display various levels of immunogenicity. For example, in the context of SARS-CoV-2, the nucleocapsid protein is considered to be the most immunogenic. However, serological antibodies against nucleocapsid protein may not convey immunity to the SARS-CoV-2 virus. Further, neutralizing antibodies (i.e., the antibodies that a patient generates that may attenuate SARS-CoV-2 spread or infectivity) may only target very specific regions of an antigen. Regarding SARS-CoV-2, the neutralizing antibodies target the NBT protein region and block the interaction of the virus with the ACE2 receptor. As such, an important aspect of the disclosure provided herein is to measure neutralizing antibodies using a flow cytometry platform for the serological testing of one or more antigens.

In one aspect, the present disclosure is directed to a composition for detecting an antigen. An antigen is a molecule or molecular structure, for example, as may be present on the outside of a pathogen (e.g., a viral or bacterial pathogen), that can be bound to an antibody, for example, an antigen-specific antibody or capture antibody as used herein. The presence of an antigen in a patient (e.g., a human patient), or the presence of an antigen in a patient that has been vaccinated, typically initiates an immune response in the patient as would be understood by one skilled in the art.

In some embodiments, the composition of the present disclosure comprises the antigen. The antigen may include any antigen known to initiate an immune response in a patient (e.g. natural infection or vaccination). In a preferred embodiment, the antigen is a severe acute respiratory syndrome coronavirus 2 (SARS-CoV-2) antigen. SARS-CoV-2 is a strain of a coronavirus that causes coronavirus disease (COVID-19) including, but not limited to, respiratory illness. SARS-CoV-2 is a positive-sense single-stranded RNA virus with a linear RNA segment. SARS-CoV-2 is a member of the subgenus Sarbecovirus. SARS-CoV-2 comprises structural proteins including, but not limited to, spike protein, nucleocapsid protein, envelope protein, membrane protein, and hemagglutinin-esterase dimer protein. SARS-CoV2 has affinity to the receptor angiotensin converting enzyme 2 (ACE2) on cells which those skilled in the art recognize as at least one mechanism for SARS-CoV-2 cell entry.

In a more preferred embodiment of the present disclosure, the SARS-CoV-2 antigen is a spike protein, a nucleocapsid protein, an envelope protein, a membrane protein, a hemagglutinin-esterase dimer protein, or an angiotensin-converting enzyme 2 (ACE2) protein. In some embodiments, the spike protein comprises a S1 protein, a S2 protein, a trimeric spike protein, or a spike glycoprotein. In further embodiments, the S1 protein comprises an N-terminal domain (NTD) or a receptor binding domain (RBD).

In some embodiments, the antigen may comprise an amino acid sequence of SEQ ID NO. 1, an amino acid sequence of SEQ ID NO. 2, an amino acid sequence of SEQ ID NO. 3, an amino acid sequence of SEQ ID NO. 4, an amino acid sequence of SEQ ID NO. 5, an amino acid sequence of SEQ ID NO. 6, an amino acid sequence of SEQ ID NO. 7, an amino acid sequence of SEQ ID NO. 8, an amino acid sequence of SEQ ID NO. 9, or an amino acid sequence of SEQ ID NO. 10.

In some embodiments, the amino acid sequence of SEQ ID NO. 1 is:

QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPGSASSVASQSIIAYTMSLGAEN
SVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLL
FNKVTLADAGFIKQYGDCLGDIAARDLICAQKFNGLTVLPPLLTDEMIAQYTSALLA
GTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDPPEAEVQID
RLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQ.

In some embodiments, the amino acid sequence of SEQ ID NO. 2 is:

MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDESKQLQQSMSSADSTQA.

In some embodiments, the amino acid sequence of SEQ ID NO. 3 is:

MYSFVSEETGTLIVNSVLLFLAFVVFLLVTLAILTALRLCAYCCNIVNV
SLVKPTVYVYSRVKNLNSSEGVPDLLV.

In some embodiments, the amino acid sequence of SEQ ID NO. 4 is:

MADNGTITVEELKQLLEQWNLVIGFLFLAWIMLLQFAYSNRNRFLYIIK
LVFLWLLWPVTLACFVLAAVYRINWVTGGIAIAMACIVGLMWLSYFVAS
FRLFARTRSMWSFNPETNILLNVPLRGTIVTRPLMESELVIGAVIIRGH
LRMAGHSLGRCDIKDLPKEITVATSRTLSYYKLGASQRVGTDSGFAAYN
RYRIGNYKLNTDHAGSNDNIALLVQ.

In some embodiments, the amino acid sequence of SEQ ID NO. 5 is:

MLIIFLFFYFCYGFNEPLNVVSHLNHDWFLFGDSRSDCNHINNLKIKNF
DYLDIHPSLCNNGKISSSAGDSIFKSFHFTRFYNYTGEGDQIIFYEGVN
FNPYHRFKCFPNGSNDVWLLNKVRFYRALYSNMAFFRYLTFVDIPYNVS
LSKFNSCKSDILSLNNPIFINYSKEVYFTLLGCSLYLVPLCLFKSNFSQ
YYYNIDTGSVYGFSNVVYPDLDCIYISLKPGSYKVSTTAPFLSLPTKAL
CFDKSKQFVPVQVVDSRWNNERASDISLSVACQLPYCYFRNSSANYVGK
YDINHGDSGFISILSGLLYNVSCISYYGVFLYDNFTSIWPYYSFGRCPT
SSIIKHPICVYDFLPIILQGILLCLALLFVVFLLFLLYNDKSH.

In some embodiments, the amino acid sequence of SEQ ID NO. 6 is:

MFIFLLFLTLTSGSDLDRCTTFDDVQAPNYTQHTSSMRGVYYPDEIFRSDTLYLTQDL
FLPFYSNVTGFHTINHTFGNPVIPFKDGIYFAATEKSNVVRGWVFGSTMNNKSQSVIII
NNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAI
LTAFSPAQDIWGTSAAAYFVGYLKPTTFMLKYDENGTITDAVDCSQNPLAELKCSVK
SFEIDKGIYQTSNFRVVPSGDVVRFPNITNLCPFGEVENATKFPSVYAWERKKISNCV
ADYSVLYNSTFFSTFKCYGVSATKLNDLCFSNVYADSFVVKGDDVRQIAPGQTGVIA
DYNYKLPDDFMGCVLAWNTRNIDATSTGNYNYKYRYLRHGKLRPFERDISNVPFSP
DGKPCTPPALNCYWPLNDYGFYTTTGIGYQPYRVVVLSFELLNAPATVCGPKLSTDL
IKNQCVNFNFNGLTGTGVLTPSSKRFQPFQQFGRDVSDFTDSVRDPKTSEILDISPCSF
GGVSVITPGTNASSEVAVLYQDVNCTDVSTAIHADQLTPAWRIYSTGNNVFQTQAGC
LIGAEHVDTSYECDIPIGAGICASYHTVSLLRSTSQKSIVAYTMSLGADSSIAYSNNTIA
IPTNFSISITTEVMPVSMAKTSVDCNMYICGDSTECANLLLQYGSFCTQLNRALSGIA
AEQDRNTREVFAQVKQMYKTPTLKYFGGFNFSQILPDPLKPTKRSFIEDLLENKVTLA
DAGFMKQYGECLGDINARDLICAQKFNGLTVLPPLLTDDMIAAYTAALVSGTATAG
WTFGAGAALQIPFAMQMAYRENGIGVTQNVLYENQKQIANQFNKAISQIQESLTTTS
TALGKLQDVVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLMSFPQAA
PHGVVFLHVTYVPSQERNFTTAPAICHEGKAYFPREGVFVFNGTSWFITQRNFFSPQII
TTDNTFVSGNCDVVIGIINNTVYDPLQPELDSFKEELDKYFKNHTSPDVDLGDISGINA
SVVNIQKEIDRLNEVAKNLNESLIDLQELGKYEQYIKWPWYVWLGFIAGLIAIVMVTI
LLCCMTSCCSCLKGACSCGSCCKFDEDDSEPVLKGVKLHYT.

In some embodiments, the amino acid sequence of SEQ ID NO. 7 is:

SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRENGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQ.

In some embodiments, the amino acid sequence of SEQ ID NO. 8 is:

VNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSS
ANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTA
GAAAYYVGYLQPRTFLLKYNENGTITDAVDCALDPLSETKCTLKS.

In some embodiments, the amino acid sequence of SEQ ID NO. 9 is:

RVQPTESIVRFPNITNLCPFGEVENATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLS
FELLHAPATVCGPKKSTNLVKNKCVNFNFNGLTGTGVLTESNKKFLPFQ
QFGRDIADTTDAVRDPQTLEILDITPCS.

In some embodiments, the amino acid sequence of SEQ ID NO. 10 is:

MSSSSWLLLSLVAVTAAQSTIEEQAKTFLDKFNHEAEDLFYQSSLASWNYNTNITEE
NVQNMNNAGDKWSAFLKEQSTLAQMYPLQEIQNLTVKLQLQALQQNGSSVLSEDK
SKRLNTILNTMSTIYSTGKVCNPDNPQECLLLEPGLNEIMANSLDYNERLWAWESWR
SEVGKQLRPLYEEYVVLKNEMARANHYEDYGDYWRGDYEVNGVDGYDYSRGQLI
EDVEHTFEEIKPLYEHLHAYVRAKLMNAYPSYISPIGCLPAHLLGDMWGRFWTNLYS
LTVPFGQKPNIDVTDAMVDQAWDAQRIFKEAEKFFVSVGLPNMTQGFWENSMLTD
PGNVQKAVCHPTAWDLGKGDFRILMCTKVTMDDFLTAHHEMGHIQYDMAYAAQP
FLLRNGANEGFHEAVGEIMSLSAATPKHLKSIGLLSPDFQEDNETEINFLLKQALTIVG
TLPFTYMLEKWRWMVFKGEIPKDQWMKKWWEMKREIVGVVEPVPHDETYCDPAS
LFHVSNDYSFIRYYTRTLYQFQFQEALCQAAKHEGPLHKCDISNSTEAGQKLFNMLR
LGKSEPWTLALENVVGAKNMNVRPLLNYFEPLFTWLKDQNKNSFVGWSTDWSPYA
DQSIKVRISLKSALGDKAYEWNDNEMYLFRSSVAYAMRQYFLKVKNQMILFGEEDV
RVANLKPRISFNFFVTAPKNVSDIIPRTEVEKAIRMSRSRINDAFRLNDNSLEFLGIQPT
LGPPNQPPVSIWLIVFGVVMGVIVVGIVILIFTGIRDRKKKNKARSGENPYASIDISKGE
NNPGFQNTDDVQTSF.

In some embodiments, the antigen as described herein is de-glycosylated prior to or after conjugation to the capture bead. A glycosylation site on a protein or antigen is understood as a site on the protein or antigen wherein a carbohydrate (i.e., a glycosyl donor) is attached to a hydroxyl or other functional group of the protein or antigen. Further, glycosylation may refer to an enzymatic process that attaches glycans to a glycosylation site on the protein or antigen. Glycosylation can be a form of co-translational or post-translational modification. As will be understood by those skilled in the art, de-glycosylation includes the removal of a carbohydrate from a hydroxyl or other functions group of the protein or antigen. Further, de-glycosylation may refer to an enzymatic process that removes glycans from a glycosylation site on the protein or antigen.

In some embodiments, the antigen as described herein is coated or tagged with biotin. As will be understood by those skilled in the art biotinylation is a chemical or enzymatic process which incorporates biotin onto a protein or antigen. Chemical biotinylation utilizes various conjugation chemistries to yield specific or nonspecific biotinylation of amines, carboxylates, sulfhydryl's and carbohydrates. Enzymatic biotinylation provides biotinylation of a specific lysine within a certain sequence of a protein or antigen by a biotin ligase. Biotin binds to streptavidin and avidin with high affinity, a fast on-rate, and high specificity, and these interactions are exploited in many areas of biotechnology to isolate biotinylated molecules of interest.

In some embodiments, the composition of the present disclosure comprises the antigen conjugated to a capture bead. As will be understood by those skilled in the art, conjugation is a type of interaction or system with connected p orbitals with delocalized electrons which in general lowers the overall energy of a molecule or molecules and increases stability of the molecule or molecules. Further, as will be understood by those skilled in the art, a capture bead is a type of bead that is capable of capturing a molecule or molecules (e.g., an antigen). In some embodiments, the capture bead is a polystyrene bead. In some embodiments, the capture bead is a streptavidin coated polystyrene bead. In further embodiments, the capture bead is coated with streptavidin. In some embodiments, the conjugation is a biotin-streptavidin conjugation.

In some embodiments, the capture bead has a size in the range from about 2.0 μm to about 12 μm, from about 3.1 μm to about 12 μm, from about 3.1 μm to about 6.9 μm, or from about 3.1 μm to about 6.8 μm. In some embodiments, the capture bead has a size in a range from about 2.0 μm to about 11 μm, from about 2.0 μm to about 10 μm, from about 2.0 μm to about 9 μm, from about 2.0 μm to about 8 μm, from about 2.0 μm to about 7 μm, from about 2.0 μm to about 6 μm, from about 2.0 μm to about 5 μm, or from about 2.0 μm to about 4.0 μm. In some embodiments, the capture bead has a size of about 2.0 μm, about 3.0 μm, about 4.0 μm, about 5.0 μm, about 6.0 μm, about 7.0 μm, about 8.0 μm, about 9.0 μm, about 10.0 μm, about 11.0 μm, or about 12.0 μm. In a preferred embodiment, the capture bead has a size of about 3.1 μm.

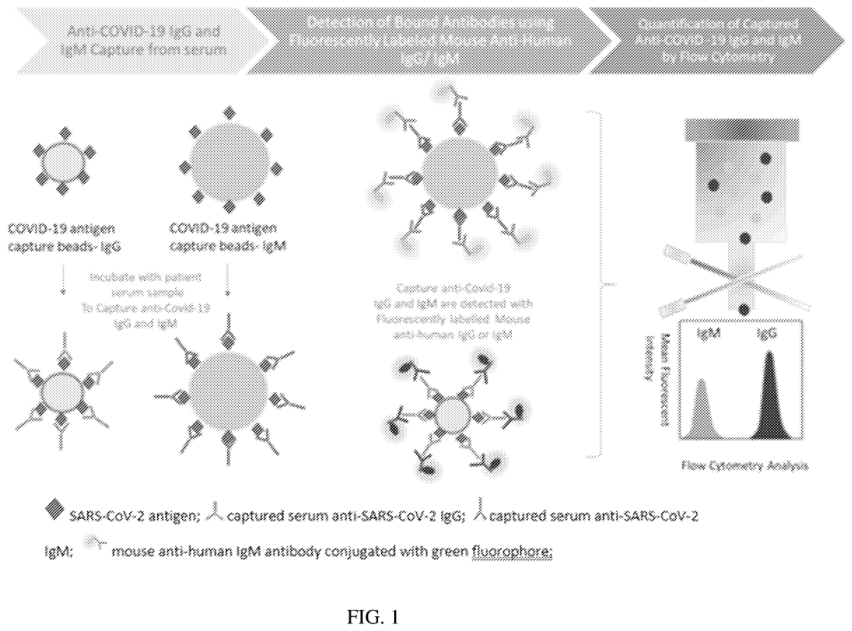

The present disclosure is further directed towards a method for detecting an infective agent in a sample from a patient by flow cytometry. In some embodiments of the present disclosure, a flow cytometry platform is used to detect an immune response (e.g., an antibody produced from a patient having an immune response to the infective agent or vaccination) in a patient sample for individuals having various disorders including, but not limited to, SARS-CoV-2. FIG. 1 depicts an illustration of an embodiment of the method of flow cytometry for detecting an infective agent such as, for example, a SARS-CoV-2 antigen.

The flow cytometry platform for detecting an infective agent disclosed herein has advantages over previously disclosed plate-based (i.e., enzyme-linked immunosorbent assay, or ELISA) serological testing including, but not limited to, increased sensitivity, reduced volume of sample required for serological testing, increased signal to noise ratio, increased efficiency in washing and removal of background signal, multiplexing capabilities of the same sample (e.g., ability to test in the sample for IgG and IgM antibodies against one or more antigens disclosed herein), increased scalability and adaptability for high throughput analysis, rapid processing and turn-around time. Further, multiplexing capabilities enables coupling with co-measurement of key cytokine profiles secreted from activated T-cells in response to exposure with SARS-CoV-2 antigens including, but not limited to, IFNγ and TNF-α. Multiplexing capabilities further enables coupling with multiple antibody profiles including, but not limited to, IgM and IgG.

In one aspect, the present disclosure is directed to a method for detecting an infective agent by flow cytometry in a sample. The method includes establishing a fluid stream. A sample from a patient having one or more antibodies is added to the fluid stream. One or more capture beads conjugated to a protein is selected. The sample and the one or more capture beads are detected by quantifying the capture antibody. The protein conjugated to the one or more capture beads is an antigen, more preferably a SARS-CoV-2 antigen.

In some embodiments, the SARS-CoV-2 antigen is a spike protein, a nucleocapsid protein, an envelope protein, a membrane protein, a hemagglutinin-esterase dimer protein, or an angiotensin-converting enzyme 2 (ACE2) protein. In some embodiments, the spike protein comprises a S1 protein, a S2 protein, a trimeric spike protein, or a spike glycoprotein. In further embodiments, the S1 protein comprises an N-terminal domain (NTD) or a receptor binding domain (RBD).

In some embodiments, the antigen may comprise an amino acid sequence of SEQ ID NO. 1, an amino acid sequence of SEQ ID NO. 2, an amino acid sequence of SEQ ID NO. 3, an amino acid sequence of SEQ ID NO. 4, an amino acid sequence of SEQ ID NO. 5, an amino acid sequence of SEQ ID NO. 6, an amino acid sequence of SEQ ID NO. 7, an amino acid sequence of SEQ ID NO. 8, an amino acid sequence of SEQ ID NO. 9, or an amino acid sequence of SEQ ID NO. 10.

In some embodiments, the antigen as described herein is de-glycosylated prior to or after conjugation to the capture bead. In some embodiments, the antigen as described herein is coated or tagged with biotin. In some embodiments, the composition of the present disclosure comprises the antigen conjugated to a capture bead. In some embodiments, the capture bead is a polystyrene bead. In some embodiments, the capture bead is a streptavidin coated polystyrene bead. In further embodiments, the capture bead is coated with streptavidin. In some embodiments, the conjugation is a biotin-streptavidin conjugation.

In some embodiments, the capture bead has a size in the range from about 2.0 μm to about 12 μm, from about 3.1 μm to about 12 μm, from about 3.1 μm to about 6.9 μm, or from about 3.1 μm to about 6.8 μm. In some embodiments, the capture bead has a size in a range from about 2.0 μm to about 11 μm, from about 2.0 μm to about 10 μm, from about 2.0 μm to about 9 μm, from about 2.0 μm to about 8 μm, from about 2.0 μm to about 7 μm, from about 2.0 μm to about 6 μm, from about 2.0 μm to about 5 μm, or from about 2.0 μm to about 4.0 μm. In some embodiments, the capture bead has a size of about 2.0 μm, about 3.0 μm, about 4.0 μm, about 5.0 μm, about 6.0 μm, about 7.0 μm, about 8.0 μm, about 9.0 μm, about 10.0 μm, about 11.0 μm, or about 12.0 μm. In a preferred embodiment, the capture bead has a size of about 3.1 μm.

In some embodiments of the method contemplated herein, the sample from a patient having one or more antibodies is from a mammal patient, or preferably from a human patient. The sample may include cells, microvesicles, blood, serum, plasma, urine, or a combination thereof from the patient. In some embodiments, the sample from the patient having one or more antibodies if from a mammal patient, or preferably a human patient, that is vaccinated with a vaccine (e.g. a SARS-CoV-2 vaccine).

In some embodiments, the sample and the one or more capture beads are incubated with a capture antibody. A capture antibody as used herein is any type of antibody capable of recognizing and/or binding to the sample from the patient having one or more antibodies. In some embodiments, the capture antibody is anti-human IgM, anti-human IgG, or a combination thereof.

In some embodiments, the sample and the one or more capture beads are incubated with a capture antibody having a detection molecule. In some embodiments, the detection molecule is a fluorophore. In some embodiments, the fluorophore is an R-Phycoerythrin protein. In further embodiments, the capture antibody is conjugated to the detection molecule.

The method for detecting an infective agent by flow cytometry in a sample may further include multiplexing the sample with a plurality of capture beads conjugated to the protein. As used herein, multiplexing is a type of assay or method used in the flow cytometry platform contemplated herein for detecting one or more antibodies in a sample from a patient using a plurality of capture beads conjugated to the protein, wherein the protein is an antigen, and wherein the antigen is a SARS-CoV-2 antigen. In some embodiments, the SARS-CoV-2 antigen is a spike protein, a nucleocapsid protein, an envelope protein, a membrane protein, a hemagglutinin-esterase dimer protein, or an angiotensin-converting enzyme 2 (ACE2) protein. In some embodiments, the spike protein comprises a S1 protein, a S2 protein, a trimeric spike protein, or a spike glycoprotein. In further embodiments, the S1 protein comprises an N-terminal domain (NTD) or a receptor binding domain (RBD). In some embodiments, the antigen may comprise an amino acid sequence of SEQ ID NO. 1, an amino acid sequence of SEQ ID NO. 2, an amino acid sequence of SEQ ID NO. 3, an amino acid sequence of SEQ ID NO. 4, an amino acid sequence of SEQ ID NO. 5, an amino acid sequence of SEQ ID NO. 6, an amino acid sequence of SEQ ID NO. 7, an amino acid sequence of SEQ ID NO. 8, an amino acid sequence of SEQ ID NO. 9, or an amino acid sequence of SEQ ID NO. 10.

The following examples are presented to demonstrate the general principles of the invention of this disclosure. The invention should not be considered as limited to the specific example presented.

### Example 1

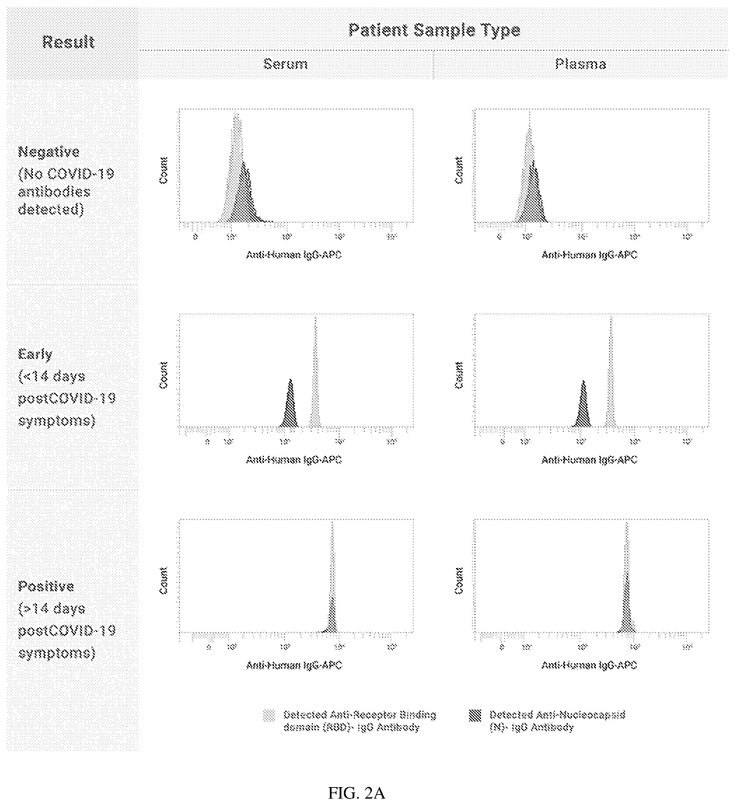

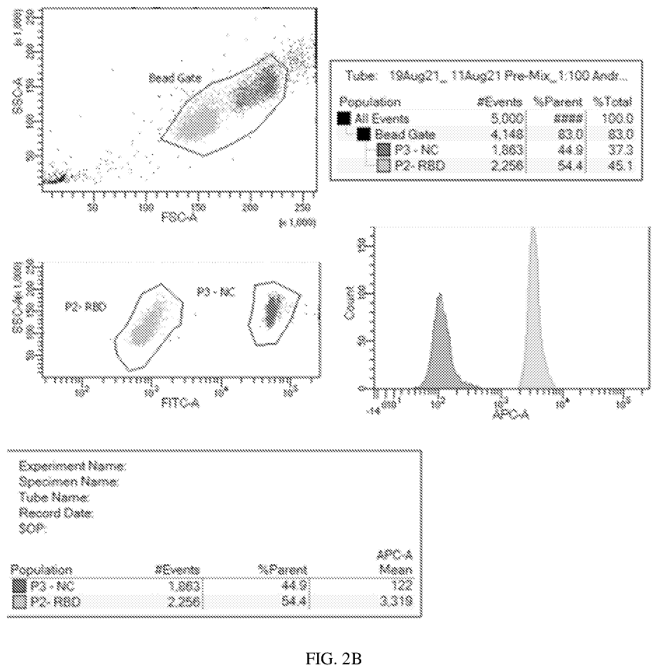

As depicted in FIGS. 2A-2B, serum of plasma from individuals (e.g. patients) is incubated with capture beads coated with either ribosome binding domain (RBD) or nucleocapsid (NC) recombinant SARS-CoV-2 virus. Anti-RBD or and-NC antibodies present in the plasma or serum are absorbed into the respective capture beads. These bound antibodies are detected with a secondary anti-human IgG antibody conjugated with the fluorophore allophycocyanin (APC).

Referencing FIG. 1A, in a negative patient (e.g. not vaccinated and with no exposure to the SARS-CoV-2 virus), there is no or low antibody levels that target the RBD or NC proteins, and therefore, there is no or low IgG antibody bound to the capture beads. The level of anti-human-IgG antibody APC conjugate that binds to the beads is therefore a background and the APC mean fluorescence intensity (MFI) signal is considered negative or background.

Further referencing FIG. 2A, post infection, we see that the patient has generated an immune response to the virus and produced Ig-G antibodies targeting both the SARS-CoV-2 virus RBD and NC proteins. The levels of the antibodies in the serum and plasma are determined by measuring the MFI on each of the two different capture beads. The MFI correlates with the amount of the anti-human IgG-APC conjugate that is bound to the anti-RBD or anti-NC present in the plasma or serum. At <14 days, we see a stronger response to the SARS-CoV-2 RBD protein, as the RBD capture bead is detected with a higher MFI signal. Two weeks after infection, we detect increased MFI signal from both the RBD and NC capture beads indicating higher levels of IgG antibodies that target these two viral proteins.

FIG. 2B depicts a post vaccination assay profile in an individual who has been vaccinated with one of the three vaccines that the Food and Drug Administration (FDA) has approved under Emergency Use Authorization (EUA), but whom has not been previously exposed to SARS-CoV-2 infection. This individual has generated antibodies that target the RBD protein (via receipt of the vaccine), but will not have levels of anti-NC antibodies above background. This is because the majority of Covid-19 vaccines implement the Spike protein of the virus to generate an immune response. The RBD protein represents a domain of the Spike protein that is involved in the entry of the virus into cells, as described herein. Therefore, antibodies targeting this region provide protection against infection. As illustrated in FIG. 2B, the APC-MFI associated with the RBD capture beads is about 30-fold higher than that associated with the NC-capture bead (e.g. background), indicating that this individual has generated high levels (e.g. titers) of anti-RBD IgG antibodies in response to vaccination.

### Example 2

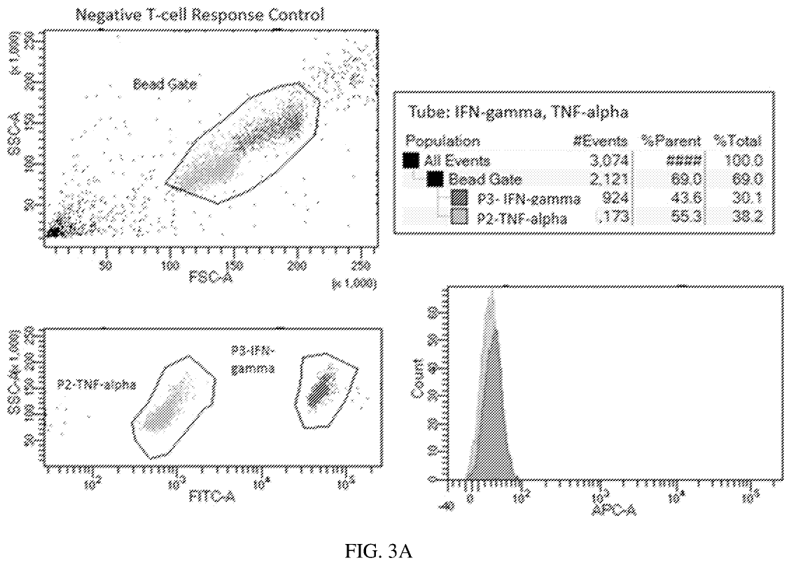

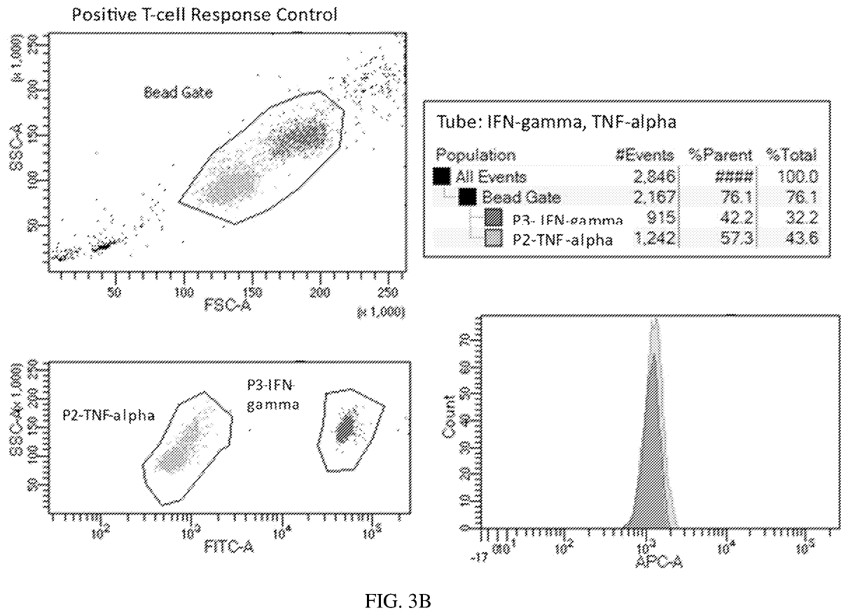

FIG. 3A depicts whole blood or peripheral blood mononuclear cells (PBMCs) recovered from an unvaccinated individual (e.g. has not received any of the Covid-19 vaccines) who has not previously contracted Covid-19 (e.g. no natural infection) were exposed in vitro to peptide pools (e.g. 1-5 μg ml⁻¹) derived from SARS-CoV-2 for 12-18 hours in an incubator at 37° C. (5% CO₂). The cells were then cultured for an additional 24 hours in the presence of 20 U ml⁻¹ of Interleukin-2 in an incubator at 37° C. (5% CO₂). The cells were then mixed with IFN-gamma and TNF-alpha capture beads (polystyrene beads of two different sizes and fluorescein isothiocyanate (FITC) intensity, coated with either anti-IFN-γ or anti-TNF-α antibodies) for 8-24 hours within an incubator at 37° C. (5% CO₂). Following incubation, the beads were recovered and washed with phosphate buffered saline (PBS) and mixed with a detection antibody (e.g. anti-IFN-γ or anti-TNF-α conjugated with a fluorophore such as APC). The sample is then acquired on the flow cytometer. The two different bead populations can be gated independently and the background signal from TNF-alpha and IFN-gamma determined. The mean fluorescent intensity (MFI) from these beads is <300 which is regarded as background.

FIG. 3B depicts whole blood or PBMCs recovered from a Covid-19 vaccinated individual or who has previously contracted Covid-19. These cells were exposed in vitro to peptide pools (1-5 μg ml⁻¹) derived from SARS-CoV-2 for 12-18 hours in an incubator at 37° C. (5% CO₂). The cells were then cultured for an additional 24 hours in the presence of 20 U ml-1 Interleukin-2 in an incubator at 37° C. (5% CO₂). The cells were then mixed with IFN-γ and TNF-α capture beads (polystyrene beads of two different sizes and FITC fluorescence, that were coated with either anti-IFN-γ or anti-TNF-α antibodies) and incubated for 8-24 hours with an incubator at 37° C. (5% CO₂). Following incubation, the beads were recovered and washed with phosphate buffered saline (PBS) and mixed with detection antibody (a different anti-IFN-γ antibody or anti-TNF-α antibody that are conjugated with a fluorophore such as APC). The sample is then acquired on the flow cytometer. The two different bead populations can be gated independently based on their FITC fluorescence signals, and they APC signal from TNF-α and IFN-γ capture beads determined the higher the APC signal MFI, the greater the level of cytokine generated by the T-cells in response to the SARS-CoV-2 antigenic peptide pool that has subsequently been captured on the beads.

1. A composition for detecting an antigen, the composition comprising the antigen conjugated to a capture bead, wherein the antigen is a severe acute respiratory syndrome coronavirus 2 (SARS-CoV-2) antigen selected from the group consisting of a spike protein, a nucleocapsid protein, an envelope protein, a membrane protein, a hemagglutinin-esterase dimer protein, and an angiotensin-converting enzyme 2 (ACE2) protein.

2. The composition of claim 1, wherein the spike protein is selected from the group consisting of a S1 protein, a S2 protein, a trimeric spike protein, and a spike glycoprotein.

3. The composition of claim 2, wherein the S1 protein comprises an N-terminal domain (NTD) or a receptor binding domain (RBD).

4. The composition of claim 1, wherein the antigen comprises an amino acid SEQ ID NO. 1, an amino acid SEQ ID NO. 2, an amino acid SEQ ID NO. 3, an amino acid SEQ ID NO. 4, an amino acid SEQ ID NO. 5, an amino acid SEQ ID NO. 6, an amino acid SEQ ID NO. 7, an amino acid SEQ ID NO. 8, an amino acid SEQ ID NO. 9, or an amino acid SEQ ID NO. 10.

5. The composition of claim 1, wherein the antigen is de-glycosylated.

6. The composition of claim 1, wherein the antigen is coated or tagged with biotin.

7. (canceled)

8. The composition of claim 1, wherein the capture bead is a streptavidin coated polystyrene bead.

9. (canceled)

10. The composition of claim 1, wherein the capture bead has a size in the range from about 2.0 μm to about 12 μm, from about 3.1 μm to about 12 μm, from about 3.1 μm to about 6.9 μm, or from about 3.1 μm to about 6.8 μm.

11. (canceled)

12. The composition of claim 1, wherein the conjugation is a biotin-streptavidin conjugation.

13. A method for detecting an infective agent by flow cytometry comprising:
establishing a fluid stream;
adding a sample from a patient having one or more antibodies;
selecting one or more capture beads conjugated to a protein;
incubating the sample and the one or more capture beads with a capture antibody having a detection molecule; and
detecting the sample and the one or more capture beads by quantifying the capture antibody, wherein the protein is an antigen, and wherein the antigen is a severe acute respiratory syndrome coronavirus 2 (SARS-CoV-2) antigen selected from the group consisting of a spike protein, a nucleocapsid protein, an envelope protein, a membrane protein, a hemagglutinin-esterase dimer protein, and an angiotensin-converting enzyme 2 (ACE2) protein.

14. The method of claim 13, wherein the spike protein is selected from the group consisting of a S1 protein, a S2 protein, a trimeric spike protein, and a spike glycoprotein.

15. (canceled)

16. The method of claim 13, wherein the antigen comprises an amino acid SEQ ID NO. 1, an amino acid SEQ ID NO. 2, an amino acid SEQ ID NO. 3, an amino acid SEQ ID NO. 4, an amino acid SEQ ID NO. 5, an amino acid SEQ ID NO. 6, an amino acid SEQ ID NO. 7, an amino acid SEQ ID NO. 8, an amino acid SEQ ID NO. 9, or an amino acid SEQ ID NO. 10.

17. The method of claim 13, wherein the antigen is de-glycosylated.

18. The method of claim 13, wherein the antigen is coated or tagged with biotin.

19. (canceled)

20. The method of claim 13, wherein the capture bead is a streptavidin coated polystyrene bead.

21. The method of claim 13, wherein the capture bead is coated with streptavidin.

22. The method of claim 13, wherein the capture bead has a size in the range from about 2.0 μm to about 12 μm, from about 3.1 μm to about 12 μm, from about 3.1 μm to about 6.9 μm, or from about 3.1 μm to about 6.8 μm.

23-26. (canceled)

27. The method of claim 13, wherein the patient is vaccinated with a SARS-CoV-2 vaccine.

28-30. (canceled)

31. The method of claim 13, wherein the capture antibody is conjugated to the detection molecule.

32. The method of claim 13, further comprising multiplexing the sample with a plurality of capture beads conjugated to the protein.


SEQUENCE LISTING (10 sequences)

SEQ ID NO: 1 - protein, 1195 aa, Artificial Sequence
  spike protein / trimeric spike protein / spike glycoprotein
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 2 - protein, 419 aa, Artificial Sequence
  nucleocapsid protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 76 aa, Artificial Sequence
  envelope protein
MYSFVSEETGTLIVNSVLLFLAFVVFLLVTLAILTALRLCAYCCNIVNVSLVKPTVYVYSRVKNLNSSEGVPDLLV

SEQ ID NO: 4 - protein, 221 aa, Artificial Sequence
  membrane protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 5 - protein, 386 aa, Artificial Sequence
  hemagglutinin-esterase dimer protein
MLIIFLFFYFCYGFNEPLNVVSHLNHDWFLFGDSRSDCNHINNLKIKNFDYLDIHPSLCNNGKISSSAGDSIFKSFHFTRFYNYTGEGDQIIFYEGVNFNPYHRFKCFPNGSNDVWLLNKVRFYRALYSNMAFFRYLTFVDIPYNVSLSKFNSCKSDILSLNNPIFINYSKEVYFTLLGCSLYLVPLCLFKSNFSQYYYNIDTGSVYGFSNVVYPDLDCIYISLKPGSYKVSTTAPFLSLPTKALCFDKSKQFVPVQVVDSRWNNERASDISLSVACQLPYCYFRNSSANYVGKYDINHGDSGFISILSGLLYNVSCISYYGVFLYDNFTSIWPYYSFGRCPTSSIIKHPICVYDFLPIILQGILLCLALLFVVFLLFLLYNDKSH

SEQ ID NO: 6 - protein, 1255 aa, Artificial Sequence
  S1 protein
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 7 - protein, 523 aa, Artificial Sequence
  S2 protein
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 8 - protein, 289 aa, Artificial Sequence
  N-terminal domain
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLK

SEQ ID NO: 9 - protein, 273 aa, Artificial Sequence
  receptor binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 10 - protein, 805 aa, Artificial Sequence [sequence omitted]